(19) United States
(12) Patent Application Publication
(10) Pub. No.: US 2007/0282021 A1
(43) Pub. Date: Dec. 6, 2007

(54) Producing ethanol and saleable organic compounds using an environmental carbon dioxide reduction process

(76) Inventor: Gregory A. Campbell, Canton, NY (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/801,157

(22) Filed: May 8, 2007

Related U.S. Application Data
(60) Provisional application No. 60/811,337, filed on Jun. 6, 2006.

(51) Int. Cl. C07C 27/26

(52) U.S. Cl. 518/726; 568/840

(57) ABSTRACT

Carbon dioxide, from a greenhouse gas source, is reacted with hydrogen, developed in an environmentally friendly process, such as from solar energy based electrolysis, to produce a mixture of ethanol and other saleable organic compounds. The ethanol and other saleable organic compounds mixture may contain substantial percentages of methanol, ethanol, and dimethylether in addition to other components which have chemical structures of predominantly carbon and hydrogen or carbon, hydrogen and oxygen. The saleable organic compounds, such as methane, methanol and ethanol may be used as a combustible fuel thus developing a green energy cycle. A portion of the organic compounds may be separated into essentially pure components and used as chemical process feed stocks. The essentially pure organic compounds may be further reacted with suitable chemical compounds to modify said organic compounds. A portion of the organic compounds may be sequestered for long or short time periods by placing them in suitable storage thus providing a mechanism for sequestering carbon dioxide and storing

### FIELD OF THE INVENTION

This invention relates to production of ethanol and other organic compounds by reaction of greenhouse gas carbon dioxide, CO₂, with green process-produced hydrogen, H₂.

### BACKGROUND OF THE INVENTION

An increasing number of scientists have concluded that the production of greenhouse gases, including carbon dioxide, is at least partly responsible for global climate change. As a result, there is strong interest is reducing and/or slowing carbon dioxide accumulation in the environment. Substantial art is available for the production of methanol from carbon dioxide, e.g., U.S. Pat. Nos. 6,894,080, 6,495,610, and 6,686,398. Also, substantial research is presently ongoing to find methods for sequestering carbon dioxide (see, e.g., U.S. Pat. No. 6,890,497) including making and storing stable hydrates. However, a substantial fraction of the energy produced pursuant to the maintenance of the world economy comes from burning fossil fuels that add carbon dioxide to the environment. Also, a significant interest in producing ethanol for use as a source of alternative fuel has recently been documented (see U.S. Pat. Nos. 6,861,248, 6,846,657, 4,952,503 as typical examples). However, most of these processes for producing an alternative fuel also produce carbon dioxide as a byproduct of the production reaction. Accordingly, there would be substantial interest in processes that are capable of producing organic compounds-based fuels including ethanol that can be operated in an environmentally benign manner, thus leading to a “green” energy cycle: organic compounds reacting with oxygen to produce carbon dioxide that is reacted with hydrogen to produce organic compounds. If desired, these organic compounds could be pumped into long term storage, such as depleted oil wells or caverns, in effect permitting sequestration of the carbon dioxide removed from the environment by the process. By putting these organic compounds in storage, the process effectively represents a mechanism for environmentally storing solar energy without the need for batteries. This would have the effect of potentially lowering the content of CO₂ in the environment, while at the same time providing a future source of carbon-based green energy when the naturally abundant crude oil, coal, tar sands, and shale oil are practically depleted.

In order to achieve the goal of reducing the accumulation of environmental carbon dioxide in accordance with the proposed process of reacting CO₂ with hydrogen to produce organic compounds, an environmentally-sound process must be used to produce hydrogen. That is, what is needed is a process that does not produce more greenhouse gases than would be converted by the process of creating organic compounds from CO₂. In such a process, the hydrogen would be produced by processes that do not result in the simultaneous release of substantial quantities of carbon dioxide. One method for producing the hydrogen is to use electrical power produced by solar collection to electrolyze water. Another method could be electrical energy from nuclear power plants for electrolysis. Still other sources of clean hydrogen production could be hydroelectric or wind power. There are other new, non-greenhouse gas producing processes that are presently in the process of development. Two such processes involve producing zinc oxide and hydrogen from zinc metal and water. It was previously discovered that it is possible to use pure zinc to extract the oxygen from water, therefore releasing hydrogen. This process can be performed at the relatively low temperature of 350° C. (662° F.). Another process is based on producing iron oxide and hydrogen from iron metal and water. See “Reduction of aqueous CO₂ at ambient temperature using zero-valent iron-based composites” Guan, G. et al, Green Chemistry , 2003, 5 , 630-634. Still another method would be to produce the hydrogen by steam reforming of organic compounds, converting the produced carbon dioxide into carbon monoxide plus hydrogen by reaction with water. A further option can involve using the Boudouard reaction and using the carbon monoxide as a reactant with other chemicals to produce higher molecular weight organic compounds and products utilizing established technology. See, e.g., U.S. Pat. No. 4,134,912. Finally, breaking the water molecules by simply heating them is not practical today because of the fact that the separation of hydrogen from oxygen requires a temperature exceeding 2,500° C. (4,532° F.). Extensive work is now ongoing to develop feasible separation technology for oxygen and hydrogen at these temperatures. If such efforts prove successful, the results could provide an excellent source of hydrogen.

It takes more energy to convert carbon dioxide to organic compounds than is available from the combustion of these compounds. This has represented a major deterrent to making ethanol and other saleable organic compounds from CO₂; the production of the necessary quantities of energy requires both equipment and a source of fuel, generally based on fossil fuel, which is a continuous process cost.

### SUMMARY OF THE INVENTION

The present invention provides processes and systems for producing organic compounds from recovered greenhouse gas carbon dioxide. In accordance with the invention the carbon dioxide is concentrated. A source of energy not based on combustion of organic compounds produces no net greenhouse gas carbon dioxide. A catalytic reactor system reacts hydrogen and carbon dioxide to produce organic compounds. Catalyst or catalysts are utilized in the catalytic reactor system. A separation system separates the reactor system products into essentially pure streams. A storage system is provided for the reaction products.

The source of energy may be a solar powered electrical system.

The source of hydrogen may be the electrolysis of water.

Greenhouse gas carbon dioxide may be removed from the air and may be concentrated with hindered amine technology.

Major products are ethanol, methanol, and other chemicals with up to twelve carbons.

The catalyst may be a mixture of iron, copper, zinc, potassium and aluminum.

The catalytic reactor system, may comprise one reactor to six reactors.

The separation system may include a stripper and distillation system.

The storage system may be storage tanks connected to a distribution system, or it may be abandoned salt mines or the like.

### BRIEF DESCRIPTION OF THE DRAWINGS

FIG. 1 provides an exemplary overview of an inventive process for producing organic compounds from a novel carbon dioxide hydrodgenation process.

FIG. 2 depicts an embodiment of the elements comprising the hydrogen source.

FIG. 3 illustrates an embodiment of the elements comprising the carbon dioxide source.

FIG. 4 shows an embodiment of the carbon monoxide utilization process.

FIG. 5 provides an embodiment of the integration of carbon dioxide sequestration and organic compound production with a coal gasification turbine electric power plant.

### DETAILED DESCRIPTION OF ILLUSTRATIVE EMBODIMENTS

In a preferred embodiment of this invention there would be little or no capital cost to collect solar energy and convert it into electric energy or high temperature thermal energy. Moreover, the solar based fuel would be essentially cost free (except for initial capital outlay and ongoing maintenance), since the sun constantly provides solar electromagnetic energy onto the earth's surface. Carbon dioxide from atmospheric sources or from combustion processes could thus be a desirable source for a carbon-based green energy cycle if the variable cost of the energy was essentially zero. The invention described below utilizes a unique combination of processes in such a manner that develops a new and unique “green” process for sequestering carbon dioxide, storing solar energy and producing combustible organic compounds that can be used as green fuels.

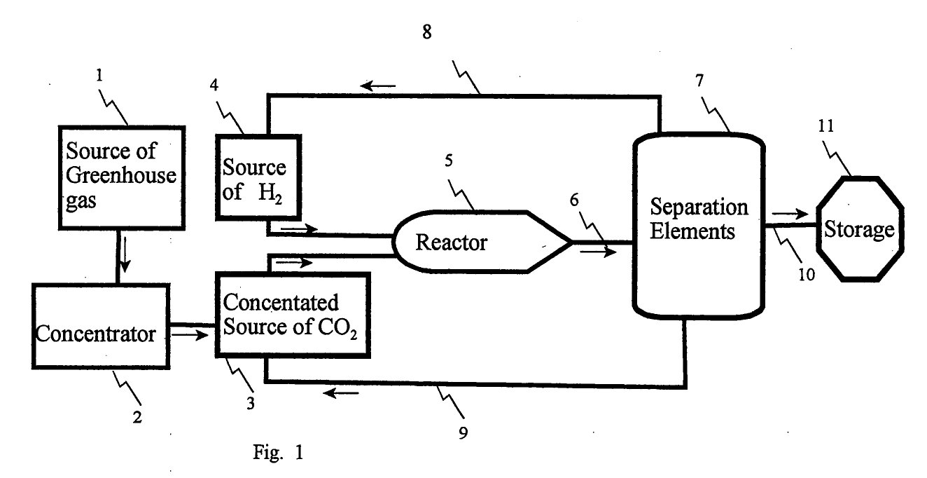

One embodiment of the overall process is shown in FIG. 1. A source of greenhouse gas 1 is concentrated in concentrator 2. Concentrated CO₂ from the source 3 is fed to reactor 5. In the preferred embodiment concentrated CO₂ is recycled from the separator to be later described. Hydrogen from source 4 is fed to reactor 6. Hydrogen is also recycled from the separator which will be subsequently described. Reactor 5 comprises the following: reactant and product containment elements, heat transfer and control elements, reactant and product flow control elements, process monitoring elements, and catalyst elements to facilitate the reaction to produce organic compounds product which is conveyed by stream 6 to separation elements 7. Separation elements 7 separate the unreacted H₂ and CO₂ from the products and recycles these gases in streams 8 and 9, respectively. The separated products are conveyed by stream 10 to storage facilities 11.

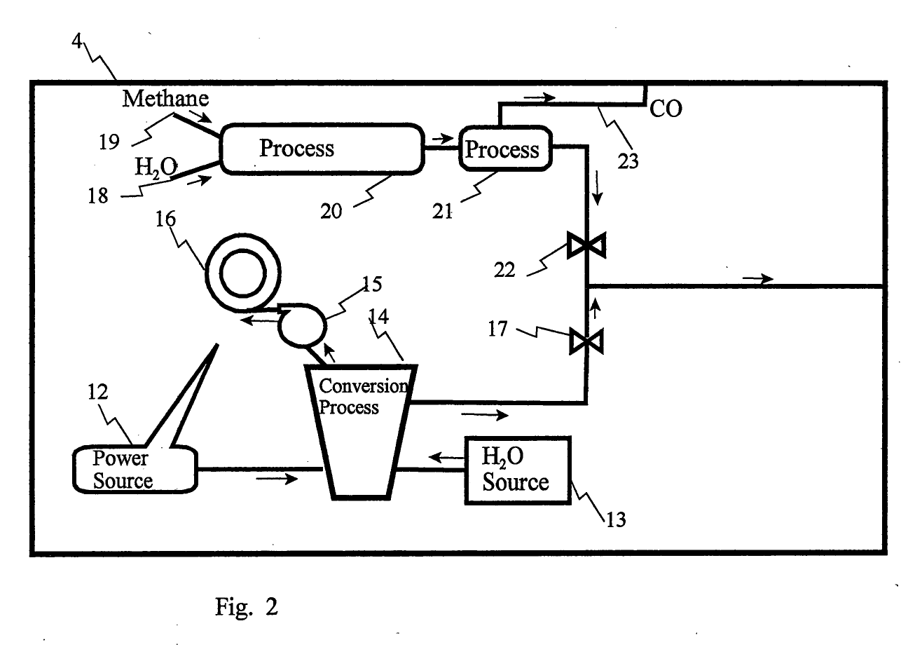

An embodiment of the hydrogen source 5 is shown in FIG. 2. Non-organic compounds are a source of electric power 12. This power source may be substantially separated from the process described in FIG. 1 and the power transported through the national electric grid. A source of water 13 is converted to H₂. The electrical power is applied to conversion process 14 where two product streams are produced. O₂ rich product is conveyed by pump or compressor 15 to storage tank 16. H₂ rich product is conveyed through control element 17 the source 4 in FIG. 1.

A second source of H₂ comprises the following: Organic compounds, such as methane, 19 and H₂O 18 are fed as the reactants in process 20, organic-conpounds-hydrogen reformer. H₂ and CO₂ are the primary products which exit process 20. The CO₂ is then further reacted in process 21, a Boudouard reactor, to produce the CO stream 23. Process 21 also contains separation elements that produce streams of essentially H₂ rich stream and essentially CO rich stream. The separated and essentially H₂ rich stream is conveyed through control element 22 to the source of H₂ (FIG. 1) from which it is fed to the reactor 5 as part of source 4. The separated and essentially CO₂ rich stream may be conveyed to other elements of the process.

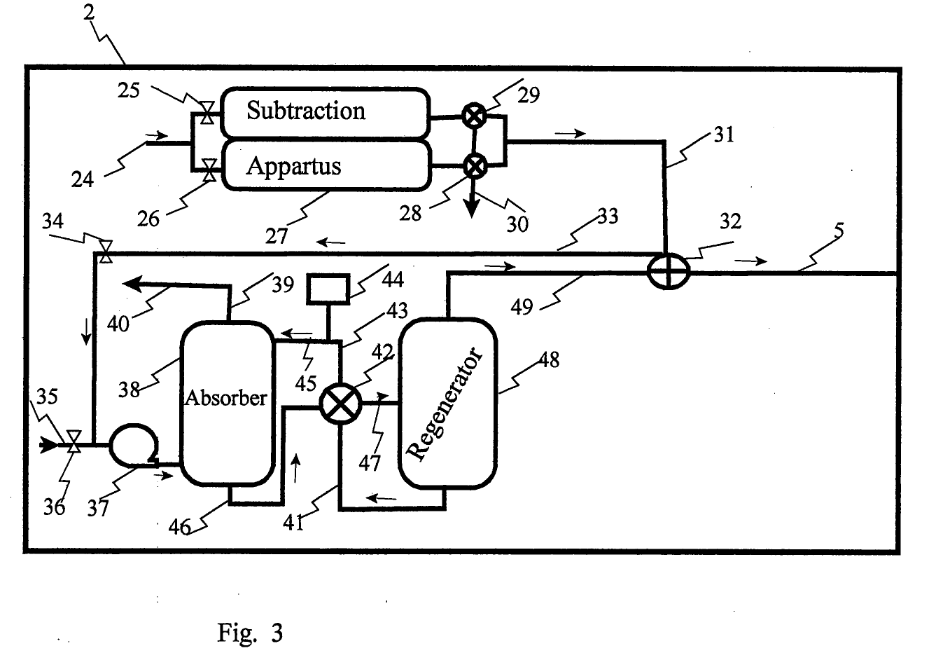

An embodiment of the CO₂ source is shown in FIG. 3. One possible source of CO₂ comprises the following elements. A low concentration stream 24 of CO₂ is in a range of 315 ppm to 10,000 ppm. This stream is conveyed by control elements 25 and 26 into process 27. One embodiment of process 27 is a CO₂ subtraction apparatus. The exit streams, one CO₂ rich and one CO₂ lean, are acted on by control elements systems 28 and 29 which coordinate the output of the subtraction apparatus such as a pressure swing separation system. The CO₂ lean stream is shown at 30. The CO₂ rich stream is shown at 31. If stream 31 contains essentially high concentration CO₂ then control element 32 directs the CO₂ rich reactant to source 3 in FIG. 1. If stream 31 has a concentration that needs to be further enriched in CO₂ it is directed by control elements 32, 33, to an enrichment process through control element 34. High or low concentration sources, containing greater than 315 ppm of CO₂, from 31 or 35 are supplied through control elements 34 or 36 and past 37 which directs them to absorber 38. The CO₂ subtracted source leaves the absorber at 39 and is returned to the atmosphere by stream 40. The active material 41 is directed in this embodiment through a heat exchanger 42 to stream 43. Elements 44, a storage vessel, provide make up of the active material and the combined materials from 43 and 44 are transported by stream 45 into absorber. The essentially reacted active material is conveyed by stream 46 to heat exchanger 42 and by stream 47 to the regenerator 48. The highly enriched CO₂, in stream 49 is directed through control element 32 to the reactor 5 feed.

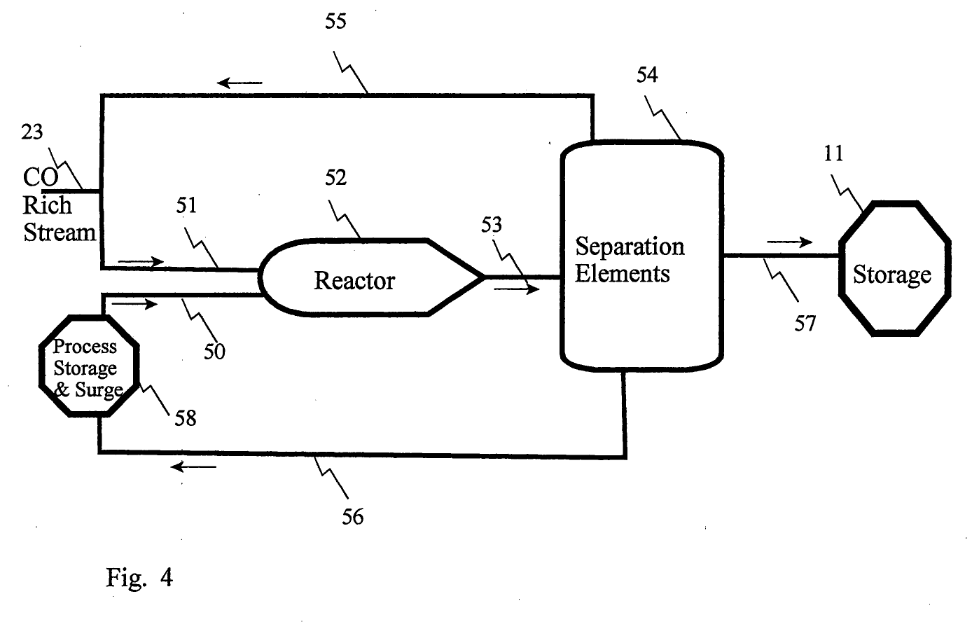

FIG. 4 shows an embodiment of the CO utilization process. The process comprises the following elements. An essentially CO rich source 23 is fed through stream 51 to reactor elements 52. A suitable feed organic compounds rich source is fed through stream 50. Reactor element 52 contains catalyst to facilitate the reaction to produce organic compounds product which is conveyed by stream 53 to separation elements 54. Separation elements 54 separate the unreacted CO and feed organic compounds from the products and recycles them using streams 55 and 56 respectively. The separated products are conveyed in stream 57 to storage facilities 11. Tank 58 provides process storage and surge for the feed organic compounds.

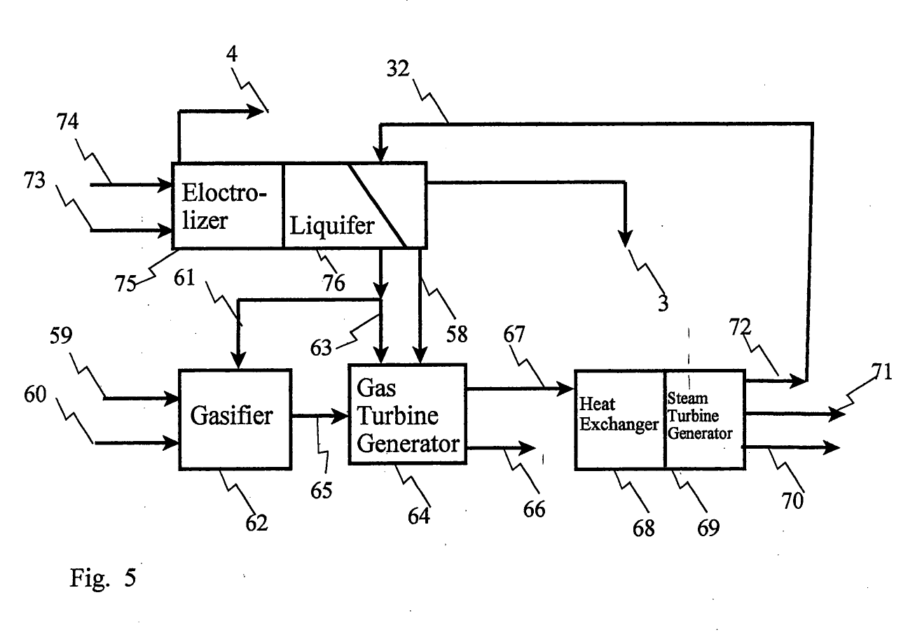

FIG. 5 shows an embodiment of the integration of carbon dioxide sequestration and organic compounds production with a coal gasification turbine electric power plant. Coal 59, water 60 and oxygen 61 are fed to a coal gasifier 62. Oxygen 63 is fed to a gas turbine electric generator 64 to burn syngas 65 producing power 66 and exhaust 67. Exhaust gas heat exchanger 68 produces high pressure steam which is fed to steam turbine electric generator 69. This process produces electric power 70 and low pressure steam 71. The cooled turbine gas 72 is sent to be concentrated in stream 32. The concentrated CO₂ is sent to the organic compounds reactor system 3. Electric power 73 and water 74 produce H₂ and O₂ in electrolizer 75. The H₂ is conveyed to the organic compounds process 4. The O₂ is liquefied in liquefier 76.

### EXAMPLE 1

A water saturated stack gas/air is fed into an Amine absorber column. In the absorber, Diethanolamine (DEA) at a strength of 30% in water is used as the absorbent. The number of stages and the operating condition of the amine absorber are listed in the tables below. Sweet gas escapes from the top of the column. Rich amine leaves the absorber and moves through a turbo-expander for stepping down its high pressure and getting work out of it. Low pressure rich amine is flashed off in a flash vessel to release off a part of the absorbed CO₂. Rich amine from the bottom of the flash vessel goes to the regenerator via a lean amine/rich amine heat exchanger where it is heated to about 90° C. The number of stages, heat load at the reboiler and the operating condition in the regenerator are changed depending upon the amount of amine flowing and its CO₂ loading. Desorbed CO₂ comes out of the condenser and is sent to the Ethanol synthesis plant. Bottom of the regenerator goes back to the absorber via a cooler. The operating conditions are as follows:
Stream Name: Air
CO₂ content: 400 ppm
Flow rate: 60000 Kgmole/hr
Lean amine flow rate: 1874 Kgmole/hr
Lean amine temperature: 35° C.
Lean amine composition:

| | CO₂ | H₂O | DEA |
|---|---|---|---|
| Mole fraction in | 0.0018 | 0.7187 | 0.2795 |
| Lean Amine | | | |

Absorber details:

| | | Tray | | | |
|---|---|---|---|---|---|
| | No | diameter | Top temp. | Column top | Bottom Pr. |
| Internals | of trays | (m) | (° C.) | pr. (kPa) | (kPa) |
| Valve tray | 20 | 5 | 37.8 | 6860 | 6880 |

Regenerator details:

| | Column | | Top | | |
|---|---|---|---|---|---|
| | dia | Reboiler | temp. | Column top | Bottom Pr. |
| Internal | (m) | duty (kJ/hr) | (° C.) | pr. (kPa) | (kPa) |
| Packed | 1.2 | 1.4 × 10⁷ | 85 | 180 | 205 |
| (structured) | | | | | |

### Net CO₂ Flow from Regenerator: 986 kg/hr

Hydrogen gas, H₂ is produced using greenhouse gas free based electrical power. Solar energy is used in this example to produce the electrical power. The power system comprises several units comprising a parabolic solar collector, a Stirling engine and an electric generator. Water electrolysis is used to split water into hydrogen and oxygen. An electrolyser is the device used for electrolysis. Water is subjected to electrical power in alkaline electrolysers and the result is hydrogen and oxygen. In the alkaline electrolyser a liquid electrolyte is used—typically a 25% potassium hydroxide solution.

The mixture of CO₂ and H₂ is passed through a heat exchanger and heated to the reaction temperature of up to about 240-260° C. The reaction mixture is passed through a series of 4 reactors with intermediate cooling by generating steam in the process. The catalyst in the reactors is composed of a mixture of Fe—Cu—Zn-AL-K, in a ratio of 1.0:0.53:0.5:2.5:0.75, respectively. The conversion of CO₂ per single pass is 28.5%. The product contains measurable quantities of alcohols with carbons from 1 to 6. The product stream is 52% ethanol and the ratio of ethanol to methanol is about 2:1. The product and unconverted reactant mixture then enters the stripper. Unconverted reactants are compressed and recycled back before reactor 5. The stripped-off ethanol goes to a distillation column and is then separated into product ethanol. A small amount of vent gas, about 1 kgmole/hr is released from the condenser of the distillation column. The final ethanol produced in this process is 87% pure with a flow of 245 kg/hr. The details of the stripper column are:

| | | Tray | | | |
|---|---|---|---|---|---|
| | No | diameter | Top temp. | Column top | Bottom Pr. |
| Internals | of trays | (m) | (° C.) | pr. (kPa) | (kPa) |
| Valve tray | 14 | 3.8 | 112 | 430 | 450 |

The details of the distillation column are:

| | No | Tray | | | |
|---|---|---|---|---|---|
| | of trays | diameter | Top temp. | Column top | Bottom Pr. |
| Internals | trays | (m) | (° C.) | pr. (kPa) | (kPa) |
| Valve tray | 20 | 3.1 | 78 | 201.3 | 221.6 |

### EXAMPLE 2

A water saturated stack gas/air is fed into the amine absorber column. In the absorber, Diethanolamine (DEA) at a strength of 30% in water is used as the absorbent. The number of stages and the operating condition of the amine absorber are listed in the tables below. Sweet gas escapes from the top of the column. Rich amine leaves the absorber and moves through a turbo-expander for stepping down its high pressure and getting work out of it. Low pressure rich amine is flashed off in a flash vessel to release off a part of the absorbed CO₂. Rich amine from the bottom of the flash vessel goes to the regenerator via a lean amine/rich amine heat exchanger where it is heated to about 90° C. The number of stages, heat load at the reboiler and the operating condition in the regenerator are changed depending upon the amount of amine flowing and its CO₂ loading. Desorbed CO₂ comes out from the condenser and is sent to the ethanol synthesis plant. Bottom of the regenerator goes back to the absorber via a cooler. The operating conditions are as follows:
Stream Name: Stack Gas
CO₂ content (vol): 8%
Flow rate: 7550 Kgmole/hr
Lean amine flow rate: 10000 Kgmole/hr
Lean amine temperature: 25° C.

### Lean Amine Composition:

| | CO₂ | H₂O | DEA |
|---|---|---|---|
| Mole fraction in | 0.0018 | 0.6587 | 0.3395 |
| Lean Amine | | | |

### Absorber Details:

| | | Tray | | | |
|---|---|---|---|---|---|
| | No | diameter | Top temp. | Column top | Bottom Pr. |
| Internals | of trays | (m) | (° C.) | pr. (kPa) | (kPa) |
| Valve tray | 22 | 2.286 | 45.8 | 6500 | 6535 |

### Regenerator Details:

| | Column | | Reboiler | Top | Column | |
|---|---|---|---|---|---|---|
| | dia | No. of | duty | temp. | top pr. | Bottom Pr. |
| Internal | (m) | trays | (kJ/hr) | (° C.) | (kPa) | (kPa) |
| Valve tray | 2.591 | 22 | 7.1 × 10⁷ | 82.5 | 155 | 195 |

### Net CO₂ Flow from Regenerator: 24561 kg/hr

Hydrogen gas, H₂ is produced using greenhouse gas free based electrical power. Solar energy is used in this example to produce the electrical power. The solar power generation is accomplished in the desert where solar energy is at a high level using an array of solar electric cells based on silicon. The electrical power is fed to the national electrical grid. Water electrolysis is used to split water into hydrogen and oxygen. An electrolyser is the device used for electrolysis. Water is subjected to electrical power removed from the grid, a substantial distance from the power generation system, in alkaline electrolysers and the result is hydrogen and oxygen. In the alkaline electrolyser a liquid electrolyte is used—typically a 25% potassium hydroxide solution.

The mixture of CO₂ and H₂ is passed through a heat exchanger and heated to the reaction temperature of 240-260° C. The reaction mixture is passed through a series of 4 reactors with intermediate cooling generating steam in the process. The catalyst in the reactors is composed of a mixture of Cu—Zn—Fe—K physically mixed with AlSiO₂. The conversion of CO₂ per single pass is 21.2%. The reactor effluent contained high levels of dimethylether, ethanol and methanol and unconverted reactant mixture then enters the stripper. Unconverted reactants are compressed and recycled back before reactor 5. Analysis of the product indicated a ethanol to methanol ratio of 9:1. The stripped-off ethanol goes to a distillation column and then is separated into product ethanol. A small amount of vent gas, about 1 kgmole/hr is released from the condenser of the distillation column. The final ethanol produced in this process is 87% pure with a flow of 13240 kg/hr. Heat energy is required in the feed preheater before reactor 5, stripper reboiler, feed preheater before distillation column and the reboiler of the distillation column. The heat required is about 280×10⁶ kJ/hr.

The energy liberated in this process is about 400×10⁶ kJ/hr. Out of this, about 300×10⁶ kJ/hr is liberated to cooling water in the stripper and distillation column condenser and the product run-down coolers. About 100×10⁶ kJ/hr can be utilized to generate steam and utilize in the process.

The details of the stripper column are:

| | No of | Tray | Top temp. | Column top | Bottom Pr. |
|---|---|---|---|---|---|
| Internals | trays | diameter (m) | (° C.) | pr. (kPa) | (kPa) |
| Valve tray | 14 | 3.8 | 112 | 430 | 450 |

The details of the distillation column are:

| | | Tray | | | |
|---|---|---|---|---|---|
| | No | diameter | Top temp. | Column top | Bottom Pr. |
| Internals | of trays | (m) | (° C.) | pr. (kPa) | (kPa) |
| Valve tray | 20 | 3.1 | 78 | 201.3 | 221.6 |

### EXAMPLE 3

A water saturated stack gas/air is fed into the amine absorber column. In the absorber, Diethanolamine (DEA) at a strength of 30% in water is used as the absorbent. The number of stages and the operating condition of the amine absorber are listed in the tables below. Sweet gas escapes from the top of the column. Rich amine leaves the absorber and moves through a turbo-expander for stepping down its high pressure and getting work out of it. Low pressure rich amine is flashed off in a flash vessel to release off a part of the absorbed CO₂. Rich amine from the bottom of the flash vessel goes to the regenerator via a lean amine/rich amine heat exchanger where it is heated to about 90° C. The number of stages, heat load at the reboiler and the operating condition in the regenerator are changed depending upon the amount of amine flowing and its CO₂ loading. Desorbed CO₂ comes out from the condenser and is sent to the ethanol synthesis plant. Bottom of the regenerator goes back to the absorber via a cooler. The operating conditions are as follows:
Stream Name: Dilute Stack Gas
CO₂ content (vol): 1%
Flow rate: 55750 Kgmole/hr
Lean amine flow rate: 10000 Kgmole/hr
Lean amine temperature: 25° C.

### Lean Amine Composition:

| | CO₂ | H₂O | DEA |
|---|---|---|---|
| Mole fraction in | 0.0018 | 0.6487 | 0.3495 |
| Lean Amine | | | |

### Absorber Details:

| | | Tray | | | |
|---|---|---|---|---|---|
| | No | diameter | Top temp. | Column top | Bottom Pr. |
| Internals | of trays | (m) | (° C.) | pr. (kPa) | (kPa) |
| Valve tray | 29 | 5.3 | 44.8 | 6860 | 6895 |

### Regenerator Details:

| | Column | | Reboiler | Top | Column | |
|---|---|---|---|---|---|---|
| | dia | No. of | duty | temp. | top pr. | Bottom Pr. |
| Internal | (m) | trays | (kJ/hr) | (° C.) | (kPa) | (kPa) |
| Valve tray | 2.59 | 22 | 7.2 × 10⁷ | 82.5 | 155 | 195 |

### Net CO₂ Flow from Regenerator: 24423 kg/hr

Hydrogen is produced using partial oxidation of methane. The products from the process are H₂ and CO. Enough H₂ is produced to react with the 24423 kg/hr of CO₂.

The mixture of CO₂ and H₂ is passed through a heat exchanger and heated to the reaction temperature of 240-260° C. The reaction mixture is passed through a series of 4 reactors with intermediate cooling by generating steam in the process. The catalyst is a mixture of Fe:Cu:Zn:Al:Pd:Ga=1.0:0.53:015:2.75:0.02:0.16. The conversion of CO₂ per single pass is 25.1%. The product, a mixture of alcohols with carbon atoms from 1 to 6, and unconverted reactant mixture then enters the stripper. Unconverted reactants are compressed and recycled back before reactor-1. The stripped-off product, ethanol which is 52% of the total product, goes to a distillation column and is then separated into product ethanol. A small amount of vent gas, about 1 kgmole/hr is released from the condenser of the distillation column. The final ethanol produced in this process is 87% pure with a flow of 13240 kg/hr. Heat energy is required in feed preheater before reactor-1, stripper reboiler, feed preheater before distillation column and the reboiler of the distillation column. The heat required is about 280×10⁶ kj/hr.

The energy liberated in this process is about 400×10⁶ kJ/hr. Out of this, about 300×10⁶ kJ/hr is liberated to cooling water in stripper and distillation column condenser and the product run-down coolers. About 100×10⁶ kJ/hr can be utilized to generate steam and utilize in the process.

The details of the stripper column are:

| | | Tray | | | |
|---|---|---|---|---|---|
| | No | diameter | Top temp. | Column top | Bottom Pr. |
| Internals | of trays | (m) | (° C.) | pr. (kPa) | (kPa) |
| Valve tray | 14 | 3.8 | 112 | 430 | 450 |

The details of the distillation column are:

| | | Tray | | | |
|---|---|---|---|---|---|
| | No | diameter | Top temp. | Column top | Bottom Pr. |
| Internals | of trays | (m) | (° C.) | pr. (kPa) | (kPa) |
| Valve tray | 20 | 3.1 | 78 | 201.3 | 221.6 |

The CO produced as a result of the H₂ production is reacted with the Methanol produced in a reactor train as a side product using a nickel catalyst to produce C₂H₆O₂. The acetic acid is purified in a separation system using distillation.

### EXAMPLE 4

A water saturated stack gas/air is fed into the amine absorber column. In the absorber, diethanolamine (DEA) at a strength of 30% in water is used as the absorbent. The number of stages and the operating condition of the amine absorber are listed in the tables below. Sweet gas escapes from the top of the column. Rich amine leaves the absorber and moves through a turbo-expander for stepping down its high pressure and getting work out of it. Low pressure rich amine is flashed off in a flash vessel to release off a part of the absorbed CO₂. Rich amine from the bottom of the flash vessel goes to the regenerator via a lean amine/rich amine heat exchanger where it is heated to about 90° C. The number of stages, heat load at the reboiler and the operating condition in the regenerator are changed depending upon the amount of amine flowing and its CO₂ loading. Desorbed CO₂ comes out from the condenser and is sent to the ethanol synthesis plant. Bottom of the regenerator goes back to the absorber via a cooler. The operating conditions are as follows:
Stream Name: Air
C0₂ content: 400 ppm
Flow rate: 60000 Kgmole/hr
Lean amine flow rate: 1874 Kgmole/hr
Lean amine temperature: 35° C.
   Lean amine composition:

| | CO₂ | H₂O | DEA |
|---|---|---|---|
| Mole fraction in | 0.0018 | 0.7187 | 0.2795 |
| Lean Amine | | | |

Absorber details:

| | | Tray | | | |
|---|---|---|---|---|---|
| | No | diameter | Top temp. | Column top | Bottom Pr. |
| Internals | of trays | (m) | (° C.) | pr. (kPa) | (kPa) |
| Valve tray | 20 | 5 | 37.8 | 6860 | 6880 |

Regenerator details:

| | Column | | Top | | |
|---|---|---|---|---|---|
| | dia | Reboiler | temp. | Column top | Bottom Pr. |
| Internal | (m) | duty (kJ/hr) | (° C.) | pr. (kPa) | (kPa) |
| Packed | 1.2 | 1.4 × 10⁷ | 85 | 180 | 205 |
| (structured) | | | | | |

### Net CO₂ Flow from Regenerator: 986 kg/hr

Hydrogen gas, H₂ is produced using greenhouse gas free process utilizing the reaction of a metal with water. Water is reacted in this case with zinc to split water into hydrogen and zinc oxide. The process is operated at 525° C.

The mixture of CO₂ and H₂ is passed through a heat exchanger and heated to the reaction temperature of 240-260° C. The reaction mixture is passed through a series of 4 reactors with intermediate cooling by generating steam in the process. The catalyst in the reactors is composed of a mixture of Fe—Cu—Zn-AL-K, in a ratio of 1.0:0.53:0.5:2.5:0.75, respectively. The conversion of CO₂ per single pass is 28.5%. The product contains measurable quantities of alcohols with carbons from 1 to 6. The product and unconverted reactant mixture then enters the stripper. Unconverted reactants are compressed and recycled back before reactor-1. The stripped-off ethanol goes to a distillation column and is then separated into product ethanol. A small amount of vent gas, about 1 kgmole/hr is released from the condenser of the distillation column. The final ethanol produced in this process is 87% pure with a flow of 245 kg/hr.

The details of the stripper column are:

| | | Tray | | | |
|---|---|---|---|---|---|
| | No | diameter | Top temp. | Column top | Bottom Pr. |
| Internals | of trays | (m) | (° C.) | pr. (kPa) | (kPa) |
| Valve tray | 14 | 3.8 | 112 | 430 | 450 |

The details of the distillation column are:

| | | Tray | | | |
|---|---|---|---|---|---|
| | No | diameter | Top temp. | Column top | Bottom Pr. |
| Internals | of trays | (m) | (° C.) | pr. (kPa) | (kPa) |
| Valve tray | 20 | 3.1 | 78 | 201.3 | 221.6 |

### EXAMPLE 5

### Preconditioning

A pressure swing system is used to increase the concentration of CO₂ in air from 377 ppm to 1% CO₂. The device comprises molecular sieves and a control system that increase the pressure on alternative columns of the molecular sieves. The pressure is then reduced and the CO₂ is recovered at a higher concentration of 10000 ppm. This is operated between 10 bar and 1 bar pressure and between ambient temperature and 250° C.
Stream Name: Concentrated Air Based
CO₂ content (vol): 1%
Flow rate: 55750 Kgmole/hr
Lean amine flow rate: 10000 Kgmole/hr
Lean amine temperature: 25° C.

### Lean Amine Composition:

| | CO₂ | H₂O | DEA |
|---|---|---|---|
| Mole fraction in | 0.0018 | 0.6487 | 0.3495 |
| Lean Amine | | | |

### Absorber Details:

| | | Tray | | | |
|---|---|---|---|---|---|
| | No | diameter | Top temp. | Column top | Bottom Pr. |
| Internals | of trays | (m) | (° C.) | pr. (kPa) | (kPa) |
| Valve tray | 29 | 5.3 | 44.8 | 6860 | 6895 |

### Regenerator Details:

| | Column | | Reboiler | Top | Column | |
|---|---|---|---|---|---|---|
| | dia | No. of | duty | temp. | top pr. | Bottom Pr. |
| Internal | (m) | trays | (kJ/hr) | (° C.) | (kPa) | (kPa) |
| Valve tray | 2.59 | 22 | 7.2 × 10⁷ | 82.5 | 155 | 195 |

### Net CO₂ Flow from Regenerator: 24423 kg/hr

Hydrogen is produced using partial oxidation of methane using a commercially available technology. The products from the process are H₂ and CO. Enough H₂ is produced to react with the 24423 kg/hr of CO₂.

The mixture of CO₂ and H₂ is passed through a heat exchanger and heated to the reaction temperature of 240-260° C. The reaction mixture is passed through a series of 4 reactors with intermediate cooling by generating steam in the process. The catalyst in the reactors is composed of a mixture of Cu—Zn—Fe—K. The conversion of CO₂ per single pass is 21.2%. The product and unconverted reactant mixture then enters the stripper. Unconverted reactants are compressed and recycled back before reactor-1. The stripped-off ethanol goes to a distillation column and is then separated into product ethanol. A small amount of vent gas, about 1 kgmole/hr is released from the condenser of the distillation column. The final ethanol produced in this process is 87% pure with a flow of 13240 kg/hr. Heat energy is required in feed preheater before reactor-1, stripper reboiler, feed preheater before distillation column and the reboiler of the distillation column. The heat required is about 280×10⁶ kJ/hr.

The energy liberated in this process is about 400×10⁶ kJ/hr. Out of this, about 300×10⁶ kJ/hr is liberated to cooling water in stripper and distillation column condenser and the product run-down coolers. About 100×10⁶ kJ/hr can be utilized to generate steam and utilize in the process.

The details of the stripper column are:

| | | Tray | | | |
|---|---|---|---|---|---|
| | No | diameter | Top temp. | Column top | Bottom Pr. |
| Internals | of trays | (m) | (° C.) | pr. (kPa) | (kPa) |
| Valve tray | 14 | 3.8 | 112 | 430 | 450 |

The details of the distillation column are:

| | | Tray | | | |
|---|---|---|---|---|---|
| | No | diameter | Top temp. | Column top | Bottom Pr. |
| Internals | of trays | (m) | (° C.) | pr. (kPa) | (kPa) |
| Valve tray | 20 | 3.1 | 78 | 201.3 | 221.6 |

Utilizing the process described in FIG.4, the CO, produced during the H₂ production, is reacted with the methanol, produced as a side product during the production of ethanol, at elevated temperature and pressure, using a nickel catalyst to produce C₂H₆O₂, acetic acid.

### EXAMPLE 6

### Preconditioning

Carbon dioxide membranes operate on the principle of selective permeation. Each gas component has a specific permeation rate. The rate of permeation is determined by the rate which a component dissolves into the carbon dioxide membrane surface and the rate at which it diffuses through the membrane. The components with higher permeation rates (such as CO₂ will permeate faster through the membrane module than components with lower permeation rates (such as N₂, O₂). For example, carbon dioxide is a “fast,” more permeable, gas than nitrogen. When a stream consisting of these two gases contacts the membrane, the carbon dioxide will permeate through the fiber at a faster rate than the nitrogen. Thus, the feed stream is separated into a air-rich (residual) stream on the exterior of the membrane fiber and a carbon dioxide-rich (permeate) stream on the interior of the membrane fiber. The primary driving force of the separation is the differential partial pressure of the permeating component. Therefore, the pressure difference between the feed gas and permeate gas and the concentration of the permeating component determine the product purity and the amount of carbon dioxide membrane surface required. This membrane separation system replaces the pressure swing system in this process in Example 5.

A membrane three stage system is used to increase the concentration of CO₂ in air from 377 ppm to 1% CO₂. This CO₂ containing gas stream is subtracted by the permeation of the CO₂ through the selective membrane. The permeate side of the membrane now contains a higher concentration of the subtracted CO₂. The CO₂ is recovered from the permeate side of the membrane at a higher concentration of 10000 ppm in the last stage of the system.
Stream Name: Concentrated Air Based
CO₂ content (vol): 1%
Flow rate: 55750 Kgmole/hr
Lean amine flow rate: 10000 Kgmole/hr
Lean amine temperature: 25° C.

### Lean Amine Composition:

| | CO₂ | H₂O | DEA |
|---|---|---|---|
| Mole fraction in | 0.0018 | 0.6487 | 0.3495 |
| Lean Amine | | | |

### Absorber Details:

| | | Tray | | | |
|---|---|---|---|---|---|
| | No | diameter | Top temp. | Column top | Bottom Pr. |
| Internals | of trays | (m) | (° C.) | pr. (kPa) | (kPa) |
| Valve tray | 29 | 5.3 | 44.8 | 6860 | 6895 |

### Regenerator Details:

| | Column | | Reboiler | Top | Column | |
|---|---|---|---|---|---|---|
| | dia | No. of | duty | temp. | top pr. | Bottom Pr. |
| Internal | (m) | trays | (kJ/hr) | (° C.) | (kPa) | (kPa) |
| Valve tray | 2.59 | 22 | 7.2 × 10⁷ | 82.5 | 155 | 195 |

### Net CO₂ Flow from Regenerator: 24423 kg/hr

Hydrogen is produced using partial oxidation of methane using a commercially available technology and by electrolysis to reduce the average cost. The products from the process are H₂ and CO. Enough H₂ is produced to react with the 24423 kg/hr of CO₂.

The mixture of CO₂ and H₂ is passed through a heat exchanger and heated to the reaction temperature of 240-260° C. The reaction mixture is passed through a series of 4 reactors with intermediate cooling by generating steam in the process. The catalyst in the reactors is composed of a mixture of Cu—Zn—Fe—K. The conversion of CO₂ per single pass is 21.2%. The product and unconverted reactant mixture then enters the stripper. Unconverted reactants are compressed and recycled back before reactor-1. The stripped-off ethanol goes to a distillation column and then separated into product ethanol. A small amount of vent gas, about 1 kgmole/hr is released from the condenser of the distillation column. The final ethanol produced in this process is 87% pure with a flow of 13240 kg/hr. Heat energy is required in the feed preheater before reactor-1, stripper reboiler, feed preheater before distillation column and the reboiler of the distillation column. The heat required is about 280×10⁶ kJ/hr.

The energy liberated in this process is about 400×10⁶ kJ/hr. Out of this, about 300×10⁶ kJ/hr is liberated to cooling water in stripper and distillation column condenser and the product run-down coolers. About 100×10⁶ kJ/hr can be utilized to generate steam and utilize in the process.

The details of the stripper column are:

| | | Tray | | | |
|---|---|---|---|---|---|
| | No | diameter | Top temp. | Column top | Bottom Pr. |
| Internals | of trays | (m) | (° C.) | pr. (kPa) | (kPa) |
| Valve tray | 14 | 3.8 | 112 | 430 | 450 |

The details of the distillation column are:

| | | Tray | Top | | |
|---|---|---|---|---|---|
| | | diameter | temp. | Column top | Bottom Pr. |
| Internals | No of trays | (m) | (° C.) | pr. (kPa) | (kPa) |
| Valve tray | 20 | 3.1 | 78 | 201.3 | 221.6 |

Utilizing the process described in FIG.4, the CO, produced during the H₂ production, is reacted with the Methanol, produced as a side product during the production of ethanol, at elevated temperature and pressure, using a nickel catalyst to produce C₂H₆O₂, acetic acid.

### EXAMPLE 7

A water saturated stack gas/air is fed into the Amine absorber column. In the absorber, diethanolamine (DEA) at a strength of 30% in water is used as the absorbent. The number of stages and the operating condition of the amine absorber are listed in the tables below. Sweet gas escapes from the top of the column. Rich amine leaves the absorber and moves through a turbo-expander for stepping down its high pressure and getting work out of it. Low pressure rich amine is flashed off in a flash vessel to release off a part of the absorbed CO₂. Rich amine from the bottom of the flash vessel goes to the regenerator via a lean amine/rich amine heat exchanger where it is heated to about 90° C. The number of stages, heat load at the reboiler and the operating condition in the regenerator are changed depending upon the amount of amine flowing and its CO₂ loading. Desorbed CO₂ comes out from the condenser and is sent to the ethanol synthesis plant. Bottom of the regenerator goes back to the absorber via a cooler. The operating conditions are as follows:
Stream Name: Stack Gas
C0₂ content (vol): 8%
Flow rate: 7550 Kgmole/hr
Lean amine flow rate: 10000 Kgmole/hr
Lean amine temperature: 25° C.

### Lean Amine Composition:

| | CO₂ | H₂O | DEA |
|---|---|---|---|
| Mole fraction in | 0.0018 | 0.6587 | 0.3395 |
| Lean Amine | | | |

### Absorber Details:

| | | Tray | | | |
|---|---|---|---|---|---|
| | No | diameter | Top temp. | Column top | Bottom Pr. |
| Internals | of trays | (m) | (° C.) | pr. (kPa) | (kPa) |
| Valve tray | 22 | 2.286 | 45.8 | 6500 | 6535 |

### Regenerator Details:

| | Column | | Reboiler | Top | Column | |
|---|---|---|---|---|---|---|
| | dia | No. of | duty | temp. | top pr. | Bottom Pr. |
| Internal | (m) | trays | (kJ/hr) | (° C.) | (kPa) | (kPa) |
| Valve tray | 2.591 | 22 | 7.1 × 10⁷ | 82.5 | 155 | 195 |

### Net CO₂ Flow from Regenerator: 24561 kg/hr

The organic compounds production process comprises: a power generation element, a hydrogen production element, a reaction element, a catalyst element, a separation element, a recycle element, and a storage element.

Hydrogen gas, H₂ is produced using greenhouse gas free based electrical power. Solar energy is used in this example to produce the electrical power. The solar power generation is accomplished in the desert where solar energy is at a high level using an array of solar collectors and thermal electric power generation. The electrical power is fed to the national electrical grid. Water electrolysis is used to split water into hydrogen and oxygen. An electrolyser is the device used for electrolysis. Water is subjected to electrical power removed from the grid, a substantial distance from the power generation system, in alkaline electrolysers and the result is hydrogen and oxygen. In the alkaline electrolyser a liquid electrolyte is used—typically a 25% potassium hydroxide solution.

The mixture of CO₂ and H₂ is passed through a heat exchanger and heated to the reaction temperature of 240-260° C. The reaction mixture is passed through a series of 4 reactors with intermediate cooling by generating steam in the process. The catalyst in the reactors is composed of a mixture of Fe—Cu—Al—K with ratios of 1.0:0.03:2.0:0.7, respectively. The conversion of CO₂ per single pass is 45.2%. The product, a mixture of organic compounds with carbon numbers from 1 to 10, and unconverted reactant mixture then enters the stripper. Unconverted reactants are compressed and recycled back before reactor-1. The stripped-off products go to storage. A fraction of the product mixture is stored to be used directly as fuel, about 25% methanol, 44% ethanol, 22% propanol, 6% butanol, 2% pentanol, and 1% hexanol. The remaining product mixture is placed in long term storage thus providing CO₂ sequestering and solar energy storage.

### EXAMPLE 8

A coal gasified power plant produces a water saturated stack gas/air which is fed to the Amine absorber column. In the absorber, diethanolamine (DEA) at a strength of 30% in water is used as the absorbent. The number of stages and the operating condition of the amine absorber are listed in the tables below. Sweet gas escapes from the top of the column. Rich amine leaves the absorber and moves through a turbo-expander for stepping down its high pressure and getting work out of it. Low pressure rich amine is flashed off in a flash vessel to release off a part of the absorbed CO₂. Rich amine from bottom of the flash vessel goes to the regenerator via a lean amine/rich amine heat exchanger where it is heated to about 90° C. The number of stages, heat load at the reboiler and the operating condition in the regenerator are changed depending upon the amount of amine flowing and its CO₂ loading. Desorbed CO₂ comes out from the condenser and is sent to the ethanol synthesis plant. Bottom of the regenerator goes back to the absorber via a cooler. The operating conditions are as follows:
Stream Name: Stack Gas
CO₂ content (vol): 8%
Flow rate: 7550 Kgmole/hr
Lean amine flow rate: 10000 Kgmole/hr
Lean amine temperature: 25° C.

### Lean Amine Composition:

| | CO₂ | H₂O | DEA |
|---|---|---|---|
| Mole fraction in | 0.0018 | 0.6587 | 0.3395 |
| Lean Amine | | | |

### Absorber Details:

| | | Tray | | | |
|---|---|---|---|---|---|
| | No | diameter | Top temp. | Column top | Bottom Pr. |
| Internals | of trays | (m) | (° C.) | pr. (kPa) | (kPa) |
| Valve tray | 22 | 2.286 | 45.8 | 6500 | 6535 |

### Regenerator Details:

| | Column | | Reboiler | Top | Column | |
|---|---|---|---|---|---|---|
| | dia | No. of | duty | temp. | top pr. | Bottom Pr. |
| Internal | (m) | trays | (kJ/hr) | (° C.) | (kPa) | (kPa) |
| Valve tray | 2.591 | 22 | 7.1 × 10⁷ | 82.5 | 155 | 195 |

### Net CO₂ Flow from Regenerator: 24561 kg/hr

Hydrogen gas, H₂ is produced using greenhouse gas free based electrical power. Solar energy is used in this example to produce the electrical power. The solar power generation is accomplished in the desert where solar energy is at a high level using solar thermal electric power generation. The electrical power is fed to the national electrical grid. Water electrolysis is used to split water into hydrogen and oxygen. An electrolyser is the device used for electrolysis. Water is subjected to electrical power removed from the national electric grid, a substantial distance from the power generation system, in an alkaline electrolyser and the result is hydrogen and oxygen. In the alkaline electrolyser a liquid electrolyte is used—typically a 25% potassium hydroxide solution. The O₂ formed in the electrolyser is liquefied and fed to the coal gasifier and the gas turbine electric power generator.

The mixture of CO₂ and H₂ is passed through a heat exchanger and heated to the reaction temperature of 240-260° C. The reaction mixture is passed through a series of 4 reactors with intermediate cooling by generating steam in the process. The catalyst in the reactors is composed of a mixture of Fe—Cu—Al—K with ratios of 1.0:0.03:2.0:0.7, respectively. The conversion of CO₂ per single pass is 45.3%. The product and unconverted reactant mixture then enters the stripper. Unconverted reactants are compressed and recycled back before reactor-1. The stripped-off ethanol goes to a distillation column and is then separated into product ethanol. A small amount of vent gas, about 1 kgmole/hr is released from the condenser of the distillation column. The final ethanol produced in this process is 87% pure with a flow of 13240 kg/hr. Heat energy is required in the feed preheater before reactor-1, stripper reboiler, feed preheater before distillation column and the reboiler of the distillation column. The heat required is about 280×10⁶ kJ/hr.

The energy liberated in this process is about 400×10⁶ kJ/hr. Out of this, about 300×10⁶ kJ/hr is liberated to cooling water in stripper and distillation column condenser and the product run-down coolers. About 100×10⁶ kJ/hr can be utilized to generate steam and utilize in the process.

The details of the stripper column are:

| | | Tray | | | |
|---|---|---|---|---|---|
| | No | diameter | Top temp. | Column top | Bottom Pr. |
| Internals | of trays | (m) | (° C.) | pr. (kPa) | (kPa) |
| Valve tray | 14 | 3.8 | 112 | 430 | 450 |

The details of the distillation column are:

| | | Tray | | | |
|---|---|---|---|---|---|
| | No | diameter | Top temp. | Column top | Bottom Pr. |
| Internals | of trays | (m) | (° C.) | pr. (kPa) | (kPa) |
| Valve tray | 20 | 3.1 | 78 | 201.3 | 221.6 |

### EXAMPLE 9

The catalyst composed of a mixture of Fe—Cu—Zn—Al—K, in a ratio of 1.0:0.53:0.5:2.5:0.75, noted in example 1 above requires the dissolution of the respective metal nitrates in a suitable aprotic solvent followed by weak acid precipitation of insoluble salts from the aprotic solvent. The metal salts of the weak acid separate into a gel phase which remains swollen by the solvent. The gel phase establishes a framework in which the remaining salts deposit during solvent evaporation. The remaining nitrates and weak organic salts decompose on slow heating in an oxidizing atmosphere to 500 C to establish a very open porous oxide framework structure. This structure in then reduced with hydrogen at 300 C to deposit an amorphous to nanocrystalline metallic Fe—Cu—Zn alloy on a potassium ferro aluminate superstructure. The catalyst is presumed to be bare metal clusters supported on the superstructure.

This catalyst has been shown to generate a variety of intermediate molecular weight products from the catalysis of a 1:3 molar ratio mixture of CO₂:H₂ heated in contact with the catalyst to 250 C. The products included both alkanes up to approximately C₁₆H₃₄ and alcohols up to approximately C₁₆H₃₃OH. The test reactions were repeated in a Parr Bomb with the same results after the fourth charge with the mixture of CO₂:H₂ as from the first run. The samples have been analyzed using a gas chromatograph mass spectrometer combination. The test runs were carried out at high a pressure and long residence time minimizing the reduction reaction to short chain alcohols.

### EXAMPLE 10

### Power Production from Cellulose Based Fuel

### Gas Turbine:

546 kg/hr of product from a cellulose pyrolisis unit at a temperature of 500° C. is fed to a gas turbine (GT). The inlet gas composition by volume is as follows: 12% CH₄, 57% CO, 21% CO₂, 5% H₂ and 5% N₂. A flow of 8500 kg/hr of ambient air is fed through a compressor (consuming 431 kW power) to the GT for the combustion. The GT generates 1405 kW of electricity. Exhaust gas from the GT at 1059° C. and 116.5 kPa flows to the superheater section of the Heat Recovery Steam Generator (HRSG). Exhaust gas at 833.7° C. enters the boiler to generate 3800 kg/hr of saturated steam at 4455 kPa pressure. The exhaust gas from the boiler passes through an economizer. It comes out at a temperature of 105° C. to pass through a cooler. The cooled flues gas enters the 1^{st} stage compressor k.o. drum. In the 1^{st} stage, the pressure is increased from 110 kPa to 525 kPa consuming 566 kW power. Due to the compression, the temperature increases to 223° C. The flue gas is cooled in the interstage cooler to 25° C. and is passed through the second stage knock out (k.o.) drum. The compressor consumes 534 kW of power and the pressure is boosted to 2300 kPa. Temperature increases to 217.3° C. which is cooled in the aftercooler.

In the HRSG , boiler feed water (BFW) pump discharge is passed through the economizer section to increase its temperature to 258° C. The steam generated from the water tube boiler is joined by the steam generated in the ethanol plant. Total steam at a flow rate of 5900 kg/hr, temp 257.9° C. and pressure of 4455 kPa enters the superheater where it is heated to 370° Cc. The superheated steam passes through a steam turbine to generate 1282 kW power. Exhaust steam is condensed in a vacuum condenser. The condensate is pumped by a vacuum pump to the deaerator. In the deaerator low pressure (LP) steam is used. BFW pump takes suction from the deaerator.

### Amine Absorber and Regenerator:

Compressed gas from the aftercooler of the second stage compressor passes through a k.o. drum before entering amine absorber. 21 m3/hr of Di-ethyl Amine (DEA) at 25° C. is used as absorbent. The tower has 26 trays. The inlet gas comprises 15.3 vol % CO₂. The lean gas exiting the tower has only 79 ppm CO₂. Rich amine from the bottom of the amine absorber passes through an expander, goes to a k.o. drum and passes through a heat exchanger before entering the regenerator tower. Regenerator has 22 trays with a top pressure of 180 kPa. 2155 kg/hr of CO₂ comes out from the regenerator top comprising 99.15% of CO₂. Lean amine from the bottom of the regenerator passes through a feed/bottom product exchanger. A pump recycles this amine (having a residual 0.15 vol % CO₂) to the absorber.

### Ethanol Plant:

CO₂ from the regenerator unit passes through a k.o. drum to the 1^{st} stage CO₂ compressor. CO₂ is compressed to 1250 kPa pressure with a power requirement of 118 kW. Compressed CO₂ passes through an intercooler to cool the gas from 217.9° C. to 85° C. The gas from the interstage k.o drum goes to the 2^{nd} stage compressor where it is compressed to 5073 kPa pressure. Compressed CO₂ enters the 1^{st} stage ethanol reactor at a temperature of 265° C. In the 1^{st} stage ethanol reactor, compressed H₂ is mixed with CO₂ for generating ethanol. H₂ is generated by electrolysis of water. O₂ generated in the process is compressed and cooled to be sold a as a by-product. H₂ is compressed to the reactor pressure by a two stage compressor. At 1^{st} stage, it is compressed to 1200 kPa pressure cooled in the interstage cooler to 35° C. and then goes to the 2^{nd} stage compressor through a k.o drum. First stage compressor consumes 305 kW power while the 2^{nd} stage consumes 236 kW power. Compressed H₂ mixes CO₂ in the reactor to generate ethanol. Product out of 1^{st} stage reactor is cooled in the reactor intercooler to 252° C. and enters the second stage reactor. The second stage intercooler reduces the temperature of the off-gas to 252° C. The exit gas out of the intercooler enters the 3rd stage reactor. The product from the 3^{rd} stage is cooled to 215° C. before passing through an expander generating 210 kW power. Steam is generated in all of the reactor intercoolers which is superheated in HRSG. The product from the expander enters the stripper which has 10 trays and the top pressure is 506.6 kPa . The reboiler duty is 1.22×10⁷ kJ/hr. The off-gas from the stripper condenser has 68.8% CO₂ and 27.7% H₂ by volume. This is recycled by a recycle gas compressor to the 1^{st} stage reactor. The compressor consumes about 190 kW power. The liquid from the stripper reboiler and the condenser are mixed and taken to the ethanol distillation column. The column has 20 trays and operates at a top pressure of 140 kPa and 85° C. The reboiler heat duty is 1.7×10⁷ kJ/hr. Bottom is pure water. From the condenser 1130 kg/hr of product is generated. The product contains 86% ethanol and 9% methanol.

As used herein, the terms “comprising” and “comprises” are to be construed as being inclusive and open rather than exclusive. Specifically, when used in this specification including the claims, the terms “comprising” and “comprises” and variations thereof mean that the specified features, steps or components are included. The terms are not to be interpreted to exclude the presence of other features, steps or components.

It will be appreciated that the above description is related to the invention by way of the example only. Many variations on the invention will be obvious to those skilled in the art and such obvious variations are within the scope of the invention as described herein whether or not expressly described.

What is claimed:

1. A method for producing ethanol and other saleable organic products from recovered greenhouse gas carbon dioxide, said method comprising:
concentrating said carbon dioxide;
producing hydrogen;
catalytically reacting said hydrogen and said carbon dioxide to produce ethanol and other saleable organic compound reaction products, the catalyst in the reaction facilitating conversion of reactants to desired ethanol and other saleable organic compound reaction products; and
separating the exit effluent from the catalytic reaction into different reactants and ethanol and other saleable organic compound reaction products.

2. The method of claim 1 wherein said hydrogen is produced from a combustion free source of energy.

3. The method of claim 2 wherein hydrogen is produced from electrolysis of water.

4. The method of claim 3 wherein the source of greenhouse gas, carbon dioxide, CO₂, has a concentration from about 300 to 500,000 ppm carbon dioxide, CO₂.

5. The method of claim 4 wherein the CO₂ is concentrated to essentially pure CO₂ with only trace amounts other components using separation elements which includes one or more of the following: membrane methods, pressure swing equipment methods, distillation methods, and hindered amine capture methods.

6. The method of claim 1 wherein the separating produces carbon dioxide which is recycled.

7. The method of claim 6 wherein the catalytic reactor system has multiple reactors and reactant recycle.

8. The method of claim 7 wherein the catalyst facilitates conversion of reactants CO₂ and H₂ into combustible saleable organic compounds.

9. The method of claim 8 wherein the catalyst comprises elements from the group: Cu—Zn—Fe—K—Al.

10. The method of claim 9 wherein each catalyst has at least three components and those components constitute 0.1 to 50 percent of the catalyst.

11. The method of claim 10 wherein combustible compounds and other saleable organic compounds are produced with up to about 20 carbon atoms per molecule.

12. The method of claim 11 wherein at least half of product combustible saleable organic compounds is made up of ethanol and methanol, with measurable minor component of other organic compounds.

13. The method of claim 11 wherein combustible compounds and other saleable organic compounds products are essentially stripped from unreacted reactants in the reactor effluent and the ethanol and other saleable organic compounds product are separated, stored and distributed.

14. The method of claim 1 wherein the source of energy is solar power.

15. The method of claim 14 wherein the catalyst comprises elements from the group: Cu—Zn—Fe—K—Al.

16. The method of claim 15 wherein each catalyst has at least three components and those components constitute 0.1 to 50 percent of the catalyst.

17. The method of claim 16 wherein combustible compounds and other saleable organic compounds are produced with up to about 20 carbon atoms per molecule.

18. The method of claim 17 wherein at least half of product combustible saleable organic compounds is made up of ethanol and methanol, with measurable minor components of other organic compounds

19. The method of claim 18 wherein combustible compounds and other saleable organic compounds products are essentially stripped from unreacted reactants in the reactor effluent and the ethanol and other saleable organic compounds product are separated, stored and distributed.

20. The method of claim 19 wherein the separation methods is distillation.

21. The method of claim 8 wherein the catalyst has components Fe:Cu:Zn:Al:Pd:Ga.

22. The method of claim 21 wherein the each catalyst component constitutes 0.01 to 4 weight ratio in the catalyst.

23. The method of claim 22 wherein combustible ethanol and other saleable organic compounds are produced with up to about 20 carbon atoms per molecule.

24. The method of claim 23 wherein at least half of product combustible saleable organic compounds is made up of ethanol and methanol, with measurable minor component of other organic compounds

25. The method of claim 24 wherein combustible compounds and other saleable organic compounds products are essentially stripped from unreacted reactants in the reactor effluent and the ethanol and other saleable organic compounds product are separated, stored and distributed.

26. The method of claim 25 wherein the separation methods is distillation.

27. An environmentally friendly method for producing ethanol and other saleable organic components from recovered greenhouse gas carbon dioxide, from a source of energy and from a hydrogen source producing carbon monoxide, comprising:
concentrating said carbon dioxide;
catalytically reacting said hydrogen and said carbon dioxide to produce ethanol and other saleable organic reaction products, the catalyst in said catalytic reaction facilitating conversion of reactants to desired ethanol and other saleable organic compounds;
separating the exit effluent into essentially different reactants and ethanol and other saleable compounds;
reacting said produced CO with organic compounds to increase the molecular weight of organic compounds and producing more saleable organic products;
storing and distributing the ethanol and other saleable organic compounds reaction products.

28. The method of claim 27 wherein the source of energy is a solar powered system.

29. The method of claim 27 wherein the source of hydrogen H₂ and CO, is from one of the addition of the processes of partial reaction of natural gas, as the Boudouard reactor, and/or electrolysis of water.

30. The method of claim 29 wherein the source of greenhouse gas carbon dioxide, CO₂, has a concentration from about 377 to 500,000 ppm carbon dioxide, CO₂.

31. The method of claim 30 wherein the CO₂ is concentrated to essentially pure CO₂ with only trace other components using appropriate system elements which includes one or more of the following: membranes methods, pressure swing equipment methods, distillation methods, and hindered amine capture methods.

32. The method of claim 27 wherein the separating produces carbon dioxide which is recycled.

33. The method of claim 32 wherein the catalytic reactor system has multiple reactors and reactant recycle.

34. The method of claim 33 wherein the catalyst facilitates conversion of reactants CO₂ and H₂ into combustible saleable organic compounds.

35. The method of claim 34 wherein the catalyst comprises elements from the group: Cu—Zn—Fe—K—Al.

36. The method of claim 35 wherein each catalyst has at least three components and those components constitute 0.1 to 50 percent of the catalyst.

37. The method of claim 36 wherein combustible compounds and other saleable organic compounds are produced with up to about 20carbon atoms per molecule.

38. The method of claim 37 wherein at least half of product combustible saleable organic compounds is made up of ethanol and methanol, with measurable minor component of other organic compounds

39. The method of claim 38 wherein combustible compounds and other saleable organic compounds products are essentially stripped from unreacted reactants in the reactor effluent and the ethanol and other saleable organic compounds product are separated, stored and distributed.

40. The method of claim 34 wherein CO is further reacted with suitable organic compounds to produce combustible saleable organic compounds.

41. The method of claim 40 wherein the combustible saleable organic compound is acetic acid.

42. An environmentally friendly system for sequestering recovered greenhouse gas carbon dioxide, CO₂, comprising:
a source of energy essentially not based on combustion of organic compounds;
a source of hydrogen, H₂, produced with said energy; a source of greenhouse gas, carbon dioxide, CO₂,
a concentrator to concentrate said carbon dioxide,
a catalytic reactor system for reaction of said hydrogen H₂, and carbon dioxide, CO₂, to produce ethanol and other saleable organic compounds reaction products;
a catalyst to be utilized in said catalytic reactor system to facilitate conversion of reactants to desired ethanol and other saleable organic compounds reaction products;
a separator to separate the reactor system exit effluent into essentially different reactants and essentially ethanol and other saleable organic compounds and reaction products effluents; and
a long term sequestering storage system for the ethanol and other saleable organic compounds reaction products.

43. The system of claim 42 wherein the source of energy is a solar powered system.

44. The system of claim 42 wherein the source of hydrogen H₂, is electrolysis of water.

45. The system of claim 44 wherein the source of greenhouse gas carbon dioxide, CO₂, has a concentration from about 377 to 500,000 ppm carbon dioxide, CO₂.

46. The system of claim 45 wherein the CO₂ is concentrated to essentially pure CO₂ with only trace other components using appropriate system elements which includes one or more of the following; membranes methods, pressure swing equipment methods, distillation methods, and hindered amine capture methods.

47. The system of claim 46 wherein the separating produces carbon dioxide which is recycled.

48. The system of claim 47 wherein the catalytic reactor system has multiple reactors and reactant recycle.

49. The system of claim 48 wherein the catalyst facilitates conversion of reactants CO₂ and H₂ into combustible saleable organic compounds.

50. The system of claim 49 wherein the catalyst comprises elements from the group: Cu—Zn—Fe—K—Al.

51. The system of claim 50 wherein each catalyst has at least three components and those components constitute 0.1 to 50 percent of the catalyst.

52. The system of claim 51 wherein combustible compounds and other saleable organic compounds are produced with up to about 20 carbon atoms per molecule.

53. The system of claim 52 wherein at least half of product combustible saleable organic compounds is made up of ethanol and methanol, with measurable minor component of other organic compounds

54. The system of claim 53 wherein combustible compounds and other saleable organic compounds products are essentially stripped from unreacted reactants in the reactor effluent and the ethanol and other saleable organic compounds product are separated, stored and distributed.

55. The system of claim 54 wherein the combustible ethanol and other saleable organic compounds products are sequestered in long term storage.

56. The system of claim 55 wherein the long term storage is depleted oil or gas wells.

57. The system of claim 55 wherein the long term storage is salt or stone caverns.

58. An environmentally friendly system for storing solar energy utilizing greenhouse gas carbon dioxide, CO₂, comprising:
a source of solar energy;
a source of hydrogen. H₂, produced with said energy;
a source of greenhouse gas, carbon dioxide, CO₂,
a concentrator to concentrate said carbon dioxide,
a catalytic reactor system for reaction of said hydrogen H₂, and carbon dioxide, CO₂, to produce ethanol and other saleable organic compounds reaction products;
a catalyst to be utilized in said catalytic reactor system to facilitate conversion of reactants to desired ethanol and other saleable organic compounds reaction products; and
a separator to separate the reactor system exit effluent into essentially different reactants and essentially ethanol and other saleable organic compounds reaction products effluents;

59. The system of claim 58 further comprising a long term storage system for the ethanol and other saleable organic compounds reaction products.

60. The system of claim 58 wherein the source of hydrogen H₂, is electrolysis of water.

61. The system of claim 58 wherein the source of greenhouse gas carbon dioxide, CO₂, has a concentration from about 377 to 500,000 ppm carbon dioxide, CO₂.

62. The system of claim 58 wherein the CO₂ is concentrated to essentially pure CO₂ with only trace other components using appropriate system elements which includes one or more of the following; membranes methods, pressure swing equipment methods, distillation methods, and hindered amine capture methods.

63. The system of claim 58 wherein the separator produces carbon dioxide which is recycled.

64. The system of claim 58 wherein the catalytic reactor system has multiple reactors and reactant recycle.

65. The system of claim 64 wherein the catalyst facilitates conversion of reactants CO₂ and H₂ into combustible saleable organic compounds.

66. The system of claim 65 wherein the catalyst comprises elements from the group: Cu—Zn—Fe—K—Al.

67. The system of claim 66 wherein each catalyst has at least three components and those components constitute 0.1 to 50 percent of the catalyst.

68. The system of claim 67 wherein combustible compounds and other saleable organic compounds are produced with up to about 20 carbon atoms per molecule.

69. The system of claim 68 wherein at least half of product combustible saleable organic compounds is made up of ethanol and methanol, with measurable minor components of other organic compounds

70. The system of claim 69 wherein combustible compounds and other saleable organic compounds products are essentially stripped from unreacted reactants in the reactor effluent and the ethanol and other saleable organic compounds product are separated, stored and distributed.

71. The system of claim 70 wherein the combustible ethanol and other saleable organic compounds products are sequestered in long term storage.

72. The system of claim 59 wherein the long term storage is depleted oil or gas wells.

73. The system of claim 59 wherein the long term storage is salt or stone caverns.
